(19) United States
(12) Patent Application Publication
(10) Pub. No.: US 2013/0029417 A1
(43) Pub. Date: Jan. 31, 2013

(54) Suppressors of RNA Silencing as Modulators of miRNA Levels

(71) Applicants: Maria Cecilia Sarmiento Guerin, Tallinn (EE); Kairi Karblane, Saku Vald (EE); Illar Pata, Tallin (EE); Erkki Truve, Tallinn (EE); Pille Pata, Tallinn (EE)

(72) Inventors: Maria Cecilia Sarmiento Guerin, Tallinn (EE); Kairi Karblane, Saku Vald (EE); Illar Pata, Tallin (EE); Erkki Truve, Tallinn (EE); Pille Pata, Tallinn (EE)

(73) Assignees: AS VAHIUURINGUTE TEHNOLOOGIA ARENDUSKESKUS, Tallinn (EE); TALLINN UNIVERSITY OF TECHNOLOGY, Tallinn (EE)

(21) Appl. No.: 13/578,703

(22) PCT Filed: Feb. 18, 2011
(86) PCT No.: PCT/EE11/00003

§ 371 Date: Aug. 13, 2012

Related U.S. Application Data
(60) Provisional application No. 61/338,529, filed on Feb. 19, 2010.

(51) Int. Cl. C12N 5/071

(52) U.S. Cl. 435/366; 435/375

(57) ABSTRACT

The invention describes use of RNA silencing suppressors or interactors of the suppressors to bring the expression of microRNAs involved in any disease, including malignant neoplasia, back to its normal level. More specifically the present invention provides a method to regulate many miRNAs at the same time. Most of the suppressors according to this invention are coded by plant viruses that unexpectedly can affect RNA silencing and modulate miRNA expression levels in mammalian cells. Also suppressors of endogenous origin are described as able to modulate miRNA expression levels.

The invention is related to the field of therapy using suppressors of RNA silencing or interactors of the suppressors to bring the expression of microRNAs involved in any disease, including malignant neoplasia, back to its normal level.

### BACKGROUND OF THE INVENTION

During the past fifteen years, our view of eukaryotic gene regulation has changed in a remarkable way, due to discoveries that revealed a novel mechanism of RNA-mediated gene silencing. RNA silencing collectively refers to the suppression of gene expression through sequence-specific interactions that are mediated by RNA (Brodersen and Voinnet, 2006). This mechanism is involved in the control of expression of endogenous genes during development and growth, maintenance of genome stability, as well as antiviral response in both animals and plants (Baulcombe, 2004; Ding and Voinnet, 2007).

Viruses and their hosts have co-evolved and this is reflected by the diverse range of viral proteins coded to counteract the RNA silencing mechanism. These proteins are known as viral suppressors of RNA silencing (Li and Ding, 2006; Ding and Voinnet, 2007). There are also negative regulators of RNA silencing coded by the host itself, known as endogenous suppressors. Up to now, few such suppressors have been described in both plants and animals (Sarmiento et al., 2006).

RNA silencing is associated with the formation of microRNAs (miRNAs), endogenous non-coding RNAs approximately 22 nucleotides in length, with a wide range of cellular functions such as differentiation and development (Reinhart et al., 2000; Grishok et al., 2001; Bernstein et al., 2003; Li and Carthew, 2005). More than 30% of the entire coding gene set is regulated by miRNAs (Lewis et al., 2005) and these are coded by 2-3% of all human genes (Alvarez-Garcia and Miska, 2005). miRNAs target predominantly transcription factors and in the case of predicted human miRNAs, more than 50% of them are localized in cancer-associated genomic regions or in fragile sites (Calin et al., 2004).

Every cellular process is likely to be regulated by miRNAs, and an aberrant miRNA expression signature is a hallmark of several diseases, including cancer. In normal cells, the expression of tumor-suppressor genes and oncogenes is tightly regulated by complex regulatory networks, where miRNAs are involved. Therefore, miRNAs can function as potential oncogenes or tumor-suppressor genes, depending on the target genes they regulate. miRNA expression profiling has provided evidence of the association of these molecules with tumor differentiation state and progression. Thus, miRNA profiles are being extensively exploited for cancer diagnosis (Lu et al., 2005, Lodes et al., 2009). Another important fact of miRNAs related to cancer is their influence in the response to anti-cancer drugs and radiation treatment. The loss or gain of miRNA function interferes with the original balance of gene expression, which may lead to treatment resistance (Weidhaas et al., 2007; Wu and Xiao, 2009).

MicroRNA misregulation is the outcome of multiple genetic and epigenetic events, which may lead to oncogenesis. The strategies used nowadays to arrange this disorder are mainly two: the use of miRNAs as drugs and the use of miRNAs as drug targets. The first strategy involves the delivery of a mature or engineered miRNA precursor in order to compensate the low dose of an miRNA acting as tumor-suppressor that is under-regulated in a certain cancer type. Mostly adenoviruses or lentiviruses expressing a specific miRNA are used in this case (Bonci et al., 2008; Kota et al., 2009), but artificial miRNAs can be also obtained from an expression vector (Liang et al., 2007).

The use of miRNAs as drug targets is the most developed strategy. In this case a specific miRNA is inhibited by strong base-pairing. Synthetic anti-miRNA oligonucleotides (AMOs) with 2′-O-methyl modification have been shown to be effective inhibitors of endogenous miRNAs (Chan et al., 2005; Si et al., 2007). One variant of these anti-miRNAs, very stable in vivo, are the so called “antagomirs”, which are chemically modified, cholesterol-conjugated, single-stranded RNA analogues, with the 2′-hydroxyl of the ribose replaced by a methoxy group and some of the phosphodiester linkages changed to phosphorothioates (Krützfeldt et al., 2005).

Another alternative are the locked nucleic acid (LNA)-based anti-miRNAs, shown to be less toxic than the previous drugs (Vester and Wengel, 2004; Elmen et al., 2008). In these analogs, the ribose ring is locked by a methylene bridge connecting the 2′-O with the 4′-C (Petersen et al., 2002). miRNA inhibition is necessary when the level of a specific miRNA that targets a tumor-suppressor gene is increased, leading to the development of a malignant tumor. All these different approaches are meant to increase or decrease the expression level of one miRNA. In some cases this may be enough to achieve a successful effect, like the regression of a liver tumor in mice (Kota et al., 2009). However, in most cases, cancer therapy seems to need the correction of the expression levels of a bunch of miRNAs simultaneously. It is hard to administer at the same time a number of molecules or a number of viruses, each targeting or expressing one miRNA. The first attempt to target many miRNAs with one construct is the use of “miRNA sponges”. These are RNA molecules with multiple miRNA binding sites that are complementary to the heptameric seeding sequence. As families of miRNAs have the same seed (2^{nd} to 8^{th} nucleotide in the miRNA sequence), then one “sponge” is able to target an entire family (Ebert et al., 2007; Loya et al., 2009). During the last two years some reports have shown that small molecules like curcumin, isoflavone, resveratol, etc. could alter miRNA expression profiles of several miRNAs, leading to the inhibition of cancer cell growth, metastasis and drug resistance (Li et al., 2009; Melkamu et al., 2010; Li et al., 2010). Most of the mentioned strategies are currently being tested in vitro and in vivo but have not reached yet any clinical trial.

### SUMMARY OF THE INVENTION

Accordingly, there is a need for a method to regulate several miRNAs simultaneously. The present invention provides a method to regulate many miRNAs at the same time, targeting the crucial misregulated miRNAs responsible for a specific cancer or other disease. This is achieved by expressing a suppressor of RNA silencing, i.e. a protein that interferes with the RNA silencing machinery producing miRNAs. In plants, suppressors of RNA silencing have been shown to change the miRNA profiles (Chapman et al., 2004; Mlotshwa et al., 2005).

This invention is related to a number of different suppressors. Most of them are proteins coded by plant viruses. Although most of these proteins have been reported suppressing RNA silencing in plants only, this invention relates to their unexpected ability to suppress RNA silencing in human cells as well. Two suppressors included in the invention are from endogenous origin (endogenous suppressors) and this invention relates to their capacity of suppressing RNA silencing in human cells.

The modulation of miRNA levels in human cells carried out by the suppressors expressed is proven by the results of miRNA expression array analysis and miRNA deep sequencing. In PC3 cancer cells the miRNA modulation was shown by miRNA expression array analysis.

Another aspect of this invention is a method to provide cure to diseases related to regulation of miRNA levels. Such method comprises modulating miRNA expression by using RNA silencing suppressors or their interactors.

Yet another aspect of this invention is a method to provide a therapy for malignant neoplasms.

### SHORT DESCRIPTION OF THE DRAWINGS

FIG. 1 shows a Western blot of HeLa cells transfected with pcDNA3.1D/V5-His-TOPO vector (Invitrogen) carrying the sequences of different RNA silencing suppressors. V5 tagged (SEQ ID NO: 8) RNA silencing suppressors were detected with anti-V5 antibody 24 hours after transfection. Expression of LacZ in a similar vector (pcDNA 3.1D/V5-His/lacZ, Invitrogen) is shown as control. Molecular masses were checked with a protein ladder. Reference numbers are on the right of the figure.

FIG. 2 shows a Western blot of ULK3 expression in HEK 293 cells cotransfected with pcDNA3.1D/V5-His-TOPO vector (Invitrogen) carrying the sequences of different RNA silencing suppressors along with a plasmid carrying FLAG tagged (SEQ ID NO:9) human ULK3 sequence (SEQ ID NO:10) and another plasmid carrying a hairpin that induces the formation of ULK3 siRNAs. ULK3 expression was detected with anti-FLAG antibody 33 hours after cotransfection. Lane 1 stands as control of ULK3 expression. Two empty vectors were cotransfected instead of the plasmids containing an RNA silencing suppressor sequence and the one aimed to produce ULK3 siRNAs. Lane 2 shows the RNA silencing of ULK3. In this case the plasmid containing an RNA silencing suppressor sequence was replaced by an empty vector (pcDNA3.1/myc-His B, Invitrogen). Lanes 3-8 show the suppressor effects of the proteins shown above the figure.

FIG. 3 shows a Western blot of prostate cancer PC-3 cells stably expressing V5 tagged RNA silencing suppressors using the anti-V5 antibody (Invitrogen). The empty vector is termed LV-iresGFP and is used as control (lane 1). Expression of the different suppressors of RNA silencing is shown in lanes 2-7. Molecular masses were checked with a protein ladder. Reference numbers are on the right of the figure.

FIG. 4a and FIG. 4b show the expression of different miRNAs in HeLa cells transfected with pcDNA3.1D/V5-His-TOPO vector (Invitrogen) carrying the sequences of different RNA silencing suppressors. Negative numbers mean down regulation of miRNA and positive numbers up regulation. The scale of fold changes represented by different positive or negative numbers is shown at the end of FIG. 4b. Different RNA silencing suppressors are shown at the top. P1 stands for RYMV P1. Empty vector pcDNA3.1/myc-His B (Invitrogen) and pcDNA 3.1D/V5-His/lacZ (Invitrogen) stand as controls (named here pcDNA and lacZ).

### DETAILED DESCRIPTION OF THE PREFERRED EMBODIEMENTS

This invention relates to a number of different RNA-silencing suppressors. Most of them are proteins coded by plant viruses. Most of these proteins have been reported to suppress RNA silencing in plants only, but this invention relates to their unexpected ability to suppress RNA silencing in human cells as well. Two of the disclosed suppressors are from endogenous origin (endogenous suppressors) and this invention also relates to their capacity of suppressing RNA silencing in human cells.

The modulation of miRNA levels in human cells due to expression of suppressors is shown by means of miRNA expression array analysis and miRNA deep sequencing. In human cancer cells PC3 the miRNA modulation due to expression of suppressors is proven by miRNA expression array analysis.

The invention also relates to methods to cure diseases related to increased or decreased miRNA levels.

### DEFINITIONS

The term “RNA silencing” refers to suppression of gene expression through sequence-specific interactions mediated by RNA.

The term “RNA silencing suppressor” or “suppressor of RNA silencing” as used herein refers to any protein, which is capable of blocking or reducing RNA silencing.

The term “endogenous suppressor” as used herein refers to suppressor of RNA silencing coded by the genome of the organism itself.

The term “interactor” as used herein refers to proteins or small chemical compounds interacting with RNA silencing suppressors. Several interactors are described in the literature: ALY proteins are known to interact with P19 (Park et al., 2004; Canto et al., 2006); TULA protein is known to interact with HsRLI (Smirnova et al., 2008); RNase L is also known to interact with HsRLI (Bisbal et al., 1995). In Drosophila eIF3 is known to be an interactor of RLI ortholog Pixie (Andersen and Leevers, 2007). In yeast interactors eIF3, eIF2, eIF5, Sup35 and Sup45 are known to interact with RLI ortholog Rli1 (Dong et al., 2004; Yarunin et al., 2005; Khoshnevis et al., 2010).

The invention is now described by means of non-limiting examples. One skilled in the art would realize that various changes can be made without departing from the core of this invention.

### Example 1

### Expression of RNA Silencing Suppressors in a Human Cell Line

HeLa cells were transfected with pcDNA3.1D/V5-His-TOPO vector (Invitrogen) carrying sequences of the different RNA silencing suppressors. RNA silencing suppressors that were used in this experiment were as follows:

P25: P25 of Potato virus X (SEQ ID NO: 1) (GenBank: ACX48434.1)

### AtRLI2: AtRLI2 of Arabidopsis (SEQ ID NO: 2) (GenBank: BAB01911.1)

RP1: P1 of Rice yellow mottle virus (SEQ ID NO: 3) (GenBank: CAI46308.1)
HsRLI: RLI of Homo sapiens (SEQ ID NO: 4) (also known as ABCE1) (GenBank: CAA53972.1)
P19: P19 of Tomato bushy stunt virus (SEQ ID NO: 5) (GenBank: NP_{—}062901.1)
CP 1: P1 of Cocksfoot mottle virus (SEQ ID NO: 6) (GenBank: ABG73617.1)
AC2: AC2 of African cassaya mosaic virus (SEQ ID NO: 7) (GenBank: AAO34428.1)

In this example and in all the following examples, one skilled in the art would realize that instead of P25 of Potato virus X, P25 of any potexvirus may be used. Instead of AC2 of African cassaya mosaic virus, AC2 of any geminivirus may be used. Instead of P1 of Rice yellow mottle virus or Cocksfoot mottle virus, P1 of any sobemovirus may be used, and instead of P19 of Tomato bushy stunt virus, P19 of any tombusvirus may be used.

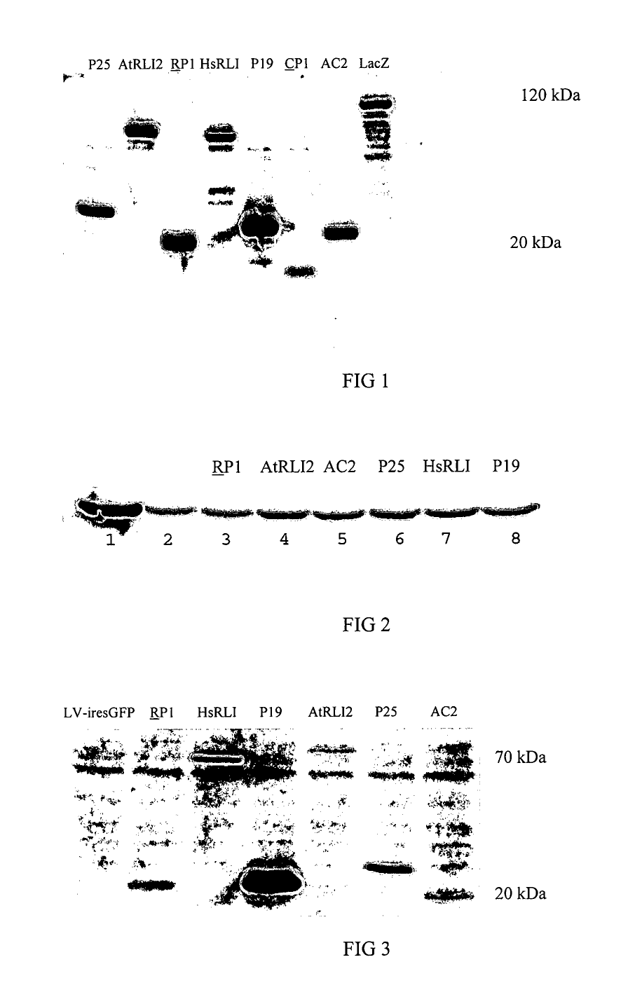

V5 tagged silencing suppressors were detected with anti-V5 antibody (Invitrogen). Western blot was carried out 24 hours after transfection using 100 μg of total protein in each case (FIG. 1).

All suppressors of RNA silencing, independently of their origin, are correctly expressed in HeLa cells. The expression levels of the different suppressors vary but are always above detection limits.

### Example 2

### Suppressor Activity of the Expressed Proteins in a Human Cell Line

HEK 293 cells were cotransfected with pcDNA3.1D/V5-His-TOPO vector (Invitrogen) carrying sequences of the different RNA silencing suppressors (SEQ ID NO: 1-7) described in Example 1 together with a plasmid (Maloverjan et al., 2010a) carrying FLAG tagged (SEQ ID NO: 9) human ULK3 sequence (SEQ ID NO: 10) and another plasmid carrying a hairpin that induces the formation of ULK3 siRNAs (Maloverjan et al., 2010b). ULK3 siRNAs induce RNA silencing of transiently expressed human ULK3 (SEQ ID NO: 10), reducing the amount of this protein. If there is suppression of ULK3 RNA silencing, then the ULK3 is not reduced in such a drastic way.

FIG. 2 is a Western blot of the transected HEK 293 cells showing ULK3 expression detected with anti-FLAG antibody 33 hours after cotransfection using 130 μg of total protein in each case. As can be seen from FIG. 2, lanes 3-8, there is a clear suppressor effect of all the proteins (RNA silencing suppressors) on ULK3 expression. The suppressor activity of HsRLI has never been reported in any cells before this disclosure.

### Example 3

### Stable Expression of RNA Silencing Suppressors in Cancer Cells Using Lentiviral Vectors

PC-3 prostate cancer cells were transduced with lentiviral vectors carrying the RNA silencing suppressor sequences (SEQ ID NO: 1-7) at multiplicity of infection greater than 1 and culti-vated for 6 days before analysis. HIV-1-based self-inactivating lentiviral vectors (LVs) were used. In LVs the expression of RNA silencing suppressors is driven from a strong constitutive promoter. This promoter also drives expression of the green fluorescent protein (GFP) via the IRES element, enabling direct monitoring of transduced cells. Lentiviral stocks were produced by transient transfection in 293FT cells essentially as described in Tiscornia et al., 2006. Expression of the different suppressors of RNA silencing is shown in lanes 2-7 of the Western blot (FIG. 3), where 130 μg of total protein were used in each case. Suppressors' names are indicated as in example 1. Molecular masses were checked with a protein ladder. Reference numbers are on the right of the figure.

All suppressors of RNA silencing, independently of their origin, are stably expressed in PC3 cells in a correct way. The expression levels of the different suppressors vary but are always above detection limits

### Example 4

### Modulation of MiRNA Expression Levels by Suppressors of RNA Silencing Expressed in Human Cells

RNA was isolated from HeLa cells transfected with the constructs described in example 1, 24 hours after transfection. Thereafter, miRNA expression array analysis was carried out with Illumina “V2 microRNA expression profiling kit” and Solexa platform was used for the deep-sequencing of cloned small RNAs (15-30 nucleotides in length). RNA from HeLa cells transfected with pcDNA3.1/myc-His B (Invitrogen) and pcDNA 3.1D/V5-His/lacZ (Invitrogen) were used as controls.

The microarray data was generated with Illumina GenomeStudio 2009.1 and gene expression module v1.1.1, considering one experiment with three technical replicates. Differential analysis was carried out applying quantile normalization, Illumina algorithm and Benjamin-Hochberg FDR methods. Significance threshold of 0.05 was used for the corrected p-values. Additionally, fold changes smaller than 0.76 and bigger than 1.24 were considered as significant (i.e. >1.24, positive or negative). The fold change in miRNA expression was calculated by 2^{(M)}, where M is the log₂-fold change after background correction and normalization.

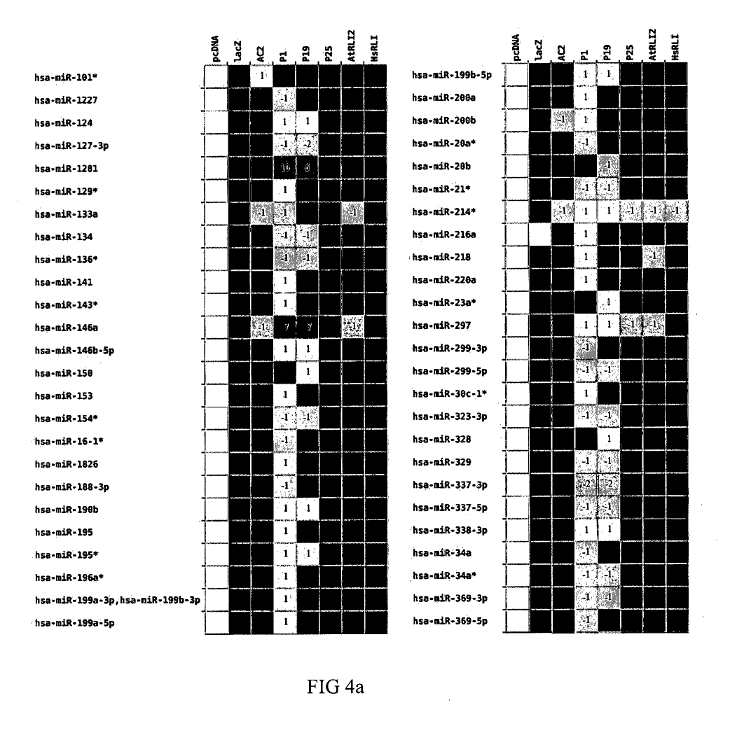

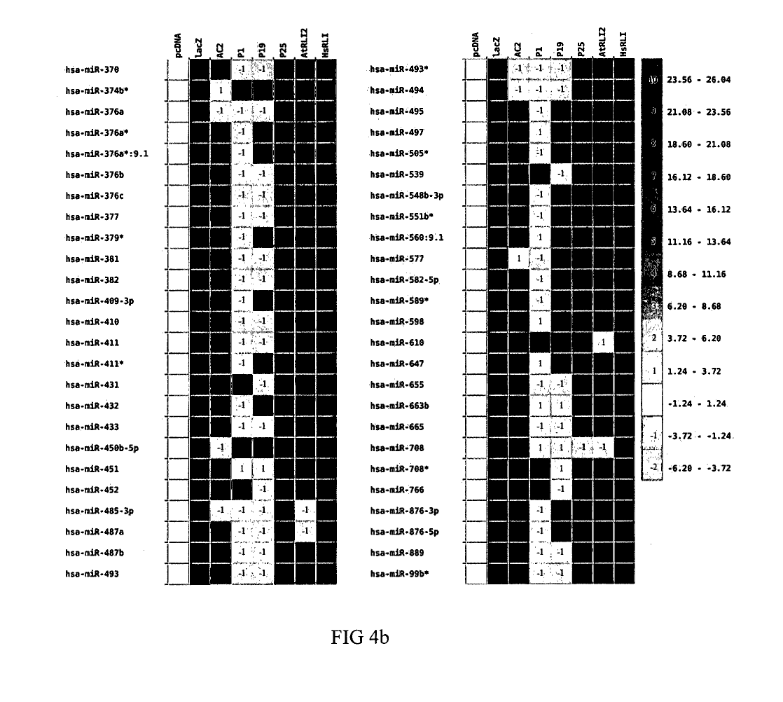

Results of the microarray analysis are shown in FIG. 4a and FIG. 4b, where white means no significant fold change, black means no statistically confident result, negative numbers mean down regulation and positive numbers mean up regulation. All tested RNA silencing suppressors induce up- or down regulation of certain miRNAs. Some of them affect less miRNA expression levels than others. In the case of the empty vector (pcDNA) there is no change in the expression levels as this was the control for the differential analysis. The expression of a protein with no RNA silencing suppressor activity (lacZ) did not affect in a statistically significant way the expression of miRNAs.

In the case of suppressor P1 from RYMV (shown as P1 in FIG. 4a and FIG. 4b), the down regulation of the following miRNAs was demonstrated with independent methods. The same results were obtained for two biological replicates with miRNA expression array analysis as well as with deep sequencing:

| miRNA ID | Adjusted P value | Fold change |
|---|---|---|
| hsa-miR-376c | 3.83E−05 | 0.39 |
| hsa-miR-493* | 0.003445 | 0.57 |
| hsa-miR-16-1* | 0.032313 | 0.69 |
| hsa-let-7f-1* | 0.035272 | 0.64 |

We conclude that in HeLa cells the RNA silencing suppressors change the levels of expression of different miRNAs, belonging to different families, at the same time.

### Example 5

### Modulation of miRNA Expression Levels by Suppressors of RNA Silencing Expressed in Cancer Cells

RNA was isolated from PC3 cells transduced with the lentiviral vectors described in example 3, one week after transduction. Thereafter, miRNA expression array analysis was carried out with Illumina “V2 microRNA expression profiling kit”. RNA from native PC3 cells was used as control.

The microarray data was generated with Illumina GenomeStudio 2009.1, considering three independent experiments with three technical replicates each. Data was normalized applying quantile normalization. Differentially expressed miRNAs were found with moderated t-test from limma library in Bioconductor. The p-values were corrected for multiple testing using False Discovery Rate (FDR). Significance threshold of 0.05 was used for the corrected p-values. Additionally, fold changes smaller than 0.8 and bigger than 1.2 were considered as significant. The fold change in miRNA expression was calculated by 2^{(M)}, where M is the log₂-fold change after background correction and normalization.

The RNA silencing suppressors change the levels of expression from different miRNAs, belonging to different families, at the same time:

| | Fold | | | miRNA | |
|---|---|---|---|---|---|
| miRNA ID | change | p-value | Suppressor | in PC | References |
| hsa-miR-374a* | 1.99 | 8.30E−06 | AC2 | ND | |
| hsa-let-7a* | 1.96 | 1.11E−08 | P19 | down//up | 1, 7//8 |
| hsa-miR-195* | 1.82 | 7.39E−07 | P19 | down//up | 1, 9//7 |
| hsa-miR-410 | 1.79 | 0.000280064 | P19 | down | 2 |
| hsa-miR-1 | 1.72 | 0.000134709 | RP1 | down | 2, 9 |
| hsa-miR-26a-2* | 1.69 | 1.81E−05 | P19 | down//up | 1, 11//2, 7 |
| hsa-miR-133a | 1.49 | 1.89E−07 | RP1 | down | 2 |
| hsa-miR-133a | 1.48 | 2.76E−07 | P19 | down | 2 |
| hsa-miR-133a | 1.45 | 8.33E−07 | P25 | down | 2 |
| hsa-miR-495 | 1.37 | 0.000125266 | P19 | ND | |
| hsa-let-7b* | 1.31 | 4.25E−05 | P19 | down | 1, 2, 10 |
| hsa-miR-221* | 1.23 | 2.26E−05 | P19 | down//up | 1, 2, 3, 4, 8, 14//5, 6, 12, 13 |
| hsa-miR-200a* | 0.79 | 0.000943193 | P19 | ND | |
| hsa-miR-1287 | 0.72 | 6.21E−06 | P19 | ND | |
| hsa-miR-1269 | 0.70 | 8.50E−05 | RP1 | ND | |
| hsa-miR-1269 | 0.69 | 3.76E−05 | P19 | ND | |
| hsa-miR-1180 | 0.66 | 0.00015814 | AC2 | ND | |
| hsa-miR-483-3p | 0.66 | 0.000190395 | RP1 | ND | |
| hsa-miR-483-3p | 0.65 | 0.000102347 | P19 | ND | |

PC: prostate cancer;
ND: no data;
ref. 1: Porkka et al., 2007;
ref. 2: Ambs et al., 2008;
ref. 3: Schafer et al., 2010;
ref. 4: Spahn et al., 2009;
ref. 5: Mercatelli et al., 2008;
ref. 6: Siva et al., 2009;
ref. 7: Volinia et al., 2006;
ref. 8: Tong et al., 2009;
ref. 9: Navon et al., 2009;
ref. 10: Ozen et al., 2008;
ref. 11: Lu et al., 2005;
ref. 12: Sun et al., 2009;
ref. 13: Galardi et al., 2007;
ref. 14: Lin et al., 2008.
Suppressors' names are as indicated in Example 1.

This table shows that changes produced by the suppressors of RNA silencing in the expression levels of the miRNAs seem to be beneficial according to published information. Column one lists the different miRNAs with up- or down regulated expression levels due to the stable expression of a suppressor of RNA silencing (shown in column 4). Fold changes (column 2) bigger than 1 show up regulation while smaller than 1 means down regulation of miRNAs. Many scientific articles have reported the up- or down regulation of specific miRNAs in the case of prostate cancer (columns 5 and 6). The comparison of the obtained fold changes (column 2) with the reported misregulation of miRNA expression levels (column 5) shows that the RNA silencing suppressors are able to correct the levels of miRNAs. If the miRNA is reported as down regulated in the case of prostate cancer, then the suppressors are up regulating it, meaning that the low miRNA level may become compensated. Therefore we suggest that RNA silencing suppressors represent a possible way of treating prostate cancer.

Based on the results represented here, the recombinant suppressors or their fragments can be used to treat malignant neoplasms. One possible way for such treatment is delivering the recombinant suppressor or fragment thereof to malignant neoplasm using cell-penetrating peptides.

### REFERENCES

Alvarez-Garcia and Miska, 2005, Development 132: 4653-4662
Ambs et al., 2008, Cancer Res. 68:6162-6170
Andersen and Leevers, 2007, J. Biol. Chem. 282: 14752-14760
Baulcombe, 2004, Nature 431: 356-363
Bernstein et al., 2003, Nat. Genet. 35: 215-217
Bisbal et al., 1995, J. Biol. Chem. 270: 13308-13317
Bonci et al., 2008, Nat. Med. 14: 1271-1277
Brodersen and Voinnet, 2006, Trends Genet. 22: 268-280
Calin et al., 2004, Proc. Natl. Acad. Sci. USA 101: 2999-3004
Canto et al., 2006, J. Virol. 80: 9064-9072
Chan et al., 2005, Nat. Rev. Genet. 6: 351-360
Chapman et al., 2004, Genes Dev. 18: 1179-1186
Ding and Voinnet, 2007, Cell 130: 413-426
Dong et al., 2004, J. Biol. Chem. 279: 42157-42168
Ebert et al., 2007, Nat. Methods 4: 721-726
Elmen et al., 2008, Nature 452: 896-899
Galardi et al., 2007, J. Biol. Chem. 282:23716-23724
Grishok et al., 2001, Cell 106: 23-34
Hayashita et al., 2005, Cancer Res. 65: 9628-9632
He et al., 2005, Nature 435: 828-833
Khoshnevis et al., 2010, EMBO Rep. 11: 214-219
Kota et al., 2009, Cell 137: 1005-1017
Krützfeldt et al., 2005, Nature 438: 685-689
Lewis et al., 2005, Cell 120: 15-20.
Li and Carthew, 2005, Cell 123: 1267-1277
Li and Ding, 2006, Annu. Rev. Microbiol. 60: 503-531
Li et al., 2009, Cancer Res. 69:6704-6712
Li et al., 2010, Pharm. Res. 27:1027-1041
Liang et al., 2007, Biochem. Biophys. Res. Commun. 363: 542-546
Lin et al., 2008, RNA 14:417-424
Lodes et al., 2009, PLOS ONE 4: e6229
Loya et al., 2009, Nat. Methods 6: 897-903
Lu et al., 2005, Nature 435: 834-838
Maloverjan et al., 2010a, Exp. Cell Res. 316: 627-637
Maloverjan et al., 2010b, J. Biol. Chem. 285: 30079-30090
Melkamu et al., 2010, Carcinogenesis 31:252-258
Mercatelli et al., 2008, PLoS One 3:e4029
Mlotshwa et al., 2005, Plant Cell 17: 2873-85
Navon et al., 2009, PloS One 4: e8003
Ozen et al., 2008, Oncogene 27:1788-1793
Park et al., 2004, Virology 323: 49-58
Petersen et al., 2002, J. Am. Chem. Soc. 124: 5974-5982
Porkka et al., 2007, Cancer Res. 67:6130-6135
Reinhart et al., 2000, Nature 403: 901-906
Sarmiento et al., 2006, Plant Mol. Biol. 61: 153-163
Schäfer et al., 2010, Int. J. Cancer 126:1166-1176
Si et al., 2007, Oncogene 26: 2799-2803
Siva et al., 2009, Mol. Cancer 8:17
Smirnova et al., 2008, Virology 372: 10-23
Spahn et al., 2009, Int. J. Cancer 127:394-403
Sun et al., 2009, Cancer Res. 69:3356-3363
Tiscornia et al., 2006, Nat. Protoc. 1: 241-245
Tong et al., 2009, Cancer Gene Ther. 16:206-216
Vester and Wengel, 2004, Biochemistry 43: 13233-13241
Volinia et al., 2006, Proc Natl Acad Sci USA 103:2257-2261
Weidhaas et al., 2007, Cancer Res. 67: 11111-11116
Wu and Xiao, 2009, Sci. China Ser. C-Life Sci. 52: 797-801
Yarunin et al., 2005, EMBO J. 24: 580-588

1. A method to modulate expression level of miRNA in mammalian cells, said method comprising:
a) providing a construct or a viral vector containing an RNA silencing (i.e. RNAi) suppressor sequence or a sequence encoding an interactor of the suppressor protein; and
b) transfecting or transducing the mammalian cells with the construct or the viral vector.

2. The method of claim 1, wherein the mammalian cell is human cell.

3. The method of claim 1, wherein the suppressor is of viral origin.

4. The method of claim 3, wherein the suppressor is selected from the group consisting of AC2 of any geminivirus, P1 of any sobemovirus, P25 of any potexvirus, and P19 of any tombusvirus.

5. The method of claim 4, wherein the suppressor is selected from the group consisting of SEQ ID NO: 1, SEQ ID NO: 3, SEQ ID NO: 5, SEQ ID NO: 6, and SEQ ID NO: 7.

6. The method of claim 1, wherein the suppressor is of endogenous origin.

7. The method of claim 6, wherein the suppressor is AtRLI2 of Arabidopsis according to SEQ ID NO: 2 or HsRLI (i.e. ABCE1) of human according to SEQ ID NO: 4.

8. The method of claim 10, wherein the interactor of the RNA silencing suppressor is a protein or a small chemical compound.

9. The method of claim 1, wherein modulation of the expression levels of miRNA is up regulation or down regulation of one or multiple miRNAs.

10. A method to treat a disease related to regulation by miRNA levels, said method comprising a step of modulating miRNA expression by use of RNA silencing suppressors or their interactors.

11. The method of claim 10, wherein the suppressor is of viral origin.

12. The method of claim 11, wherein the suppressor is selected from the group consisting of AC2 of any geminivirus, P1 of any sobemovirus, P25 of any potexvirus, and P19 of any tombusvirus.

13. The method of claim 12, wherein the suppressor is selected from the group consisting of SEQ ID NO: 1, SEQ ID NO: 3, SEQ ID NO: 5, SEQ ID NO: 6, and SEQ ID NO: 7.

14. The method of claim 10, wherein the suppressor is of endogenous origin.

15. The method of claim 14, wherein the suppressor is AtRLI2 of Arabidopsis according to SEQ ID NO: 2 or HsRLI (i.e. ABCE1) of human according to SEQ ID NO: 4.

16. The method of claim 10, wherein the disease is a cancerous disease.

17. The method of claim 16, wherein the disease is prostate cancer.

18. The method of claim 10, wherein the method comprises delivering the suppressor or a fragment thereof or its interactors into malignant neoplasm.

19. The method of claim 18, wherein the delivering is by use of cell-penetrating peptides.

20. A method to simultaneously modulate expression level of multiple miRNAs in mammalian cells, said method comprising expressing an RNA silencing suppressor sequence of viral or endogenous origin in the mammalian cell.


SEQUENCE LISTING (10 sequences)

SEQ ID NO: 1 - protein, 226 aa, Potato virus X
MDILISSLKSLGYSRTSKSLDSGPLVVHAVAGAGKSTALRKLILRHPTFTVHTLGVPDKVSIRTRGIQKPGPIPEGNFAILDEYTLDNTTRNSNQALFADPYQAPEFSLEPHFYLETSFRVPRKVADLIAGCGFDFETNSPEEGHLEITGIFKGPLLGKVIAIDEESETTLSRHGVEFVKPCQVTGLEFKVVTIVSAAPIEEIGQSTAFYNAITRSKGLTYVRAGP

SEQ ID NO: 2 - protein, 603 aa, Arabidopsis thaliana
MSDRLTRIAIVSEDRCKPKKCRQECKKSCPVVKTGKLCIEVGSTSKSAFISEELCIGCGICVKKCPFEAIQIINLPKDLAKDTTHRYGANGFKLHRLPIPRPGQVLGLVGTNGIGKSTALKILAGKLKPNLGRFNTPPDWEEILTHFRGSELQSYFIRVVEENLKTAIKPQHVDYIKEVVRGNLGKMLEKLDERGLMEEICADMELNQVLEREARQVSGGELQRFAIAAVFVKKADIYMFDEPSSYLDVRQRLKAAQVIRSLLRHDSYVIVVEHDLSVLDYLSDFVCCLYGKPGAYGVVTLPFSVREGINVFLAGFIPTENLRFRDESLTFRVSETTQENDGEVKSYARYKYPNMTKQLGDFKLEVMEGEFTDSQIIVMLGENGTGKTTFIRMLAGAFPREEGVQSEIPEFNVSYKPQGNDSKRECTVRQLLHDKIRDACAHPQFMSDVIRPLQIEQLMDQVVKTLSGGEKQRVAITLCLGKPADIYLIDEPSAHLDSEQRITASKVIKRFILHAKKTAFIVEHDFIMATYLADRVIVYEGQPAVKCIAHSPQSLLSGMNHFLSHLNITFRRDPTNFRPRINKLESIKDKEQKTAGSYYYLDD

SEQ ID NO: 3 - protein, 157 aa, Rice yellow mottle virus
MTRLEVLIRPTEQTVAKANAVGYTHTLTWVWHSQTWDVDSVNDPVLRADFDPNRSGWVAVSFACTQCTAHYYTCEQVKFFTNIPPIHYDVVCADCERRVQQDDEIDREHQERNAEISACNARALSEGKPASLVYLSRDACDIPEHSGTCRYDKYLNF

SEQ ID NO: 4 - protein, 599 aa, Homo sapiens
MADKLTRIAIVNHDKCKPKKCRQECKKSCPVVRMGKLCIEVTPQSKIAWISETLCIGCGICIKKCPFGALSIVNLPSNLEKETTHRYCANAFKLHRLPIPRPGEVLGLVGTNGIGKSAALKILAGKQKPNLGKYDDPPDWQEILTYFRGSELQNYFTKILEDDLKAIIKPQYVARFLRLAKGTVGSILDRKDETKTQAIVCQQLDLTHLKERNVEDLSGGELQRFACAVVCIQKADIFMFDEPSSYLDVKQRLKAAITIRSLINPDRYIIVVEHDLSVLDYLSDFICCLYGVPSAYGVVTMPFSVREGINIFLDGYVPTENLRFRDASLVFKVAETANEEEVKKMCMYKYPGMKKKMGEFELAIVAGEFTDSEIMVMLGENGTGKTTFIRMLAGRLKPDEGGEVPVLNVSYKPQKISPKSTGSVRQLLHEKIRDAYTHPQFVTDVMKPLQIENIIDQEVQTLSGGELQRVRLRLCLGKPADVYLIDEPSAYLDSEQRLMAARVVKRFILHAKKTAFVVEHDFIMATYLADRVIVFDGVPSKNTVANSPQTLLAGMNKFLSQLEITFRRDPNNYRPRINKLNSIKDVEQKKSGNYFFLDD

SEQ ID NO: 5 - protein, 172 aa, Tomato bushy stunt virus
MERAIQGNDAREQANSERWDGGSGGTTSPFKLPDESPSWTEWRLHNDETNSNQDNPLGFKESWGFGKVVFKRYLRYDRTEASLHRVLGSWTGDSVNYAASRFFGFDQIGCTYSIRFRGVSITVSGGSRTLQHLCEMAIRSKQELLQLAPIEVESNVSRGCPEGTETFEKESE

SEQ ID NO: 6 - protein, 108 aa, Cocksfoot mottle virus
MCEPPPGFITVQCYTSDDLLTGDSTIVKSIPVRSCFFRQGVEVVLFRCESNKHRWLKIRGPVSLTVHCDICEFRETVEIPSLPKGFKVSSDFSYSVTWNCCYSRGRTE

SEQ ID NO: 7 - protein, 135 aa, African cassava mosaic virus
MQSSSPSQNHSTQVPIKVSHRQFKKRAIRRRRVDLVCGCSYYLHINCSNHGFTHRGTHHCSSSNEWRVYLGNKQSPVFHNNQAPTTTIPAEPGHHNSPGSIQSQPAEGAGDSQMFSQLQVLDALKASDWSFLKGL

SEQ ID NO: 8 - protein, 14 aa, artificial sequence
  chemically synthesized
GKPIPNPLLGLDST

SEQ ID NO: 9 - protein, 8 aa, artificial sequence
  chemically synthesized
DYKDDDDK

SEQ ID NO: 10 - protein, 472 aa, Homo sapiens
MAGPGWGPPRLDGFILTERLGSGTYATVYKAYAKKDTREVVAIKCVAKKSLNKASVENLLTEIEILKGIRHPHIVQLKDFQWDSDNIYLIMEFCAGGDLSRFIHTRRILPEKVARVFMQQLASALQFLHERNISHLDLKPQNILLSSLEKPHLKLADFGFAQHMSPWDEKHVLRGSPLYMAPEMVCQRQYDARVSLWSMGVILYEALFGQPPFASRSFSELEEKIRSNRVIELPLRPLLSRDCRDLLQRLLERDPSRRISFQDFFAHPWVDLEHMPSGESLGRATALVVQAVKKDQEGDSAAALSLYCKALDFFVPALHYEVDAQRKEAIKAKVGQYVSRAEELKAIVSSSNQALLRQGTSARDLLREMARDKPRLLAALEVASAAMAKEEAAGGEQDALDLYQHSLGELLLLLAAEPPGRRRELLHTEVQNLMARAEYLKEQVKMRESRWEADTLDKEGLSESVRSSCTLQ